(19) United States
(12) Patent Application Publication
(10) Pub. No.: US 2012/0190833 A1
(43) Pub. Date: Jul. 26, 2012

(54) METHOD OF SELECTION, BY TWO-DIMENSIONAL SEPARATION, OF NUCLEIC ACIDS THAT BIND TO A TARGET WITH HIGH AFFINITY

(75) Inventor: Andreas Kage, Berlin (DE)

(73) Assignee: APTARES AG, Luckenwalde (DE)

(21) Appl. No.: 13/217,535

(22) Filed: Aug. 25, 2011

Related U.S. Application Data
(63) Continuation of application No. 11/948,591, filed on Nov. 30, 2007, now abandoned.
Application No. 11/948,591 is a continuation of application No. 10/398,436, filed on Sep. 22, 2003, now abandoned.

(30) Foreign Application Priority Data
Oct. 3, 2000 (DE) 10048944.3

(51) Int. Cl. C07H 21/04; C12P 19/34; C07H 1/06

(52) U.S. Cl. 536/23.1; 536/25.4; 435/91.2

(57) ABSTRACT

The invention relates to a method of selection, by two-dimensional separation, of nucleic acids that bind to a target molecule with high affinity from a mixture of nucleic acids, including the following steps: a) subjecting the mixture of nucleic acids to a physico-chemical separation step, thereby obtaining a set of mixed fractions containing the nucleic acids, a run parameter window being associated with every mixed fraction containing the nucleic acids, b) contacting a mixed fraction containing the nucleic acids with the target molecule, thereby obtaining a binding mixture containing nucleic acid/target molecule complexes, c) subjecting the binding mixture from step b) to the same physico-chemical separation step as in step a), thereby selecting nucleic acid/target molecule complexes whose run parameters are outside of the run parameter window.

### STATEMENT OF RELATED APPLICATION

This is a continuation of application Ser. No. 11/948,591, filed Nov. 30, 2007, which is a continuation of U.S. patent application Ser. No. 10/398,436, filed Sep. 22, 2003, entitled “Method of Selection, By Two-Dimensional Separation, Of Nucleic Acids That Bind To A Target With High Affinity,” which is a 371 of PCT/DE01/03818, filed Oct. 2, 2001, entitled “Method of Selection, By Two-Dimensional Separation, Of Nucleic Acids That Bind To A Target With High Affinity.” Both of the prior applications are incorporated by reference herein in their entireties.

### FIELD OF THE INVENTION

The invention relates to a method of selection, by two-dimensional separation, of nucleic acids that bind to a target with high affinity, wherein a mixture of nucleic acids is contacted with one or several defined target molecules and wherein nucleic acids that bind to the target molecule are separated from nucleic acids that do not bind.

Nucleic acids are poly or oligonucleotides with a nucleotide count of 5 to 200, in particular 20 to 200. These may be DNAs, RNAs or PNAs. In particular, the nucleic acids may be chemically derivatized, for instance by 2′ and/or 5 substitution, and/or provided with reporter molecules (molecules which permit a detection with conventional detection methods). The nucleic acid may be single or double-stranded. A target molecule may, in principle, be of any type, as far as it is not such a nucleic acid which enters into Crick/Watson base pair bonds with the nucleic acid contacted therewith. Examples for target molecules are: plastic materials, ceramics, peptides, proteins, enzymes, oligo-saccharides, polysaccharides, nucleic acids not entering into Crick/Watson bonds, lipids, but also hormones and other organic compounds, such as pheromones. Targets may also be parts of cells and complete cells, such as complete viruses and bacteria. Nucleic acids binding to non-nucleic acids are designated as aptamers, but also nucleic acids entering into non-Crick/Watson bonds with other nucleic acids. The term binding means in this invention noncovalent bindings. The last-mentioned aptamers may for instance be used for the detection of certain gene defects and/or deletions in genes. The term binding means in this invention noncovalent bindings. The term affinity relates in this invention to the binding force within complexes of the antigen/antibody type. The binding force is quantifiable by the affinity constant, which is defined under the law of mass action. In this invention, the term affinity however not only relates to the complexes with a binding site, but also to complexes having several binding sites, i.e. also comprises the term avidity. The avidity results from the number and the binding force of every binding site for multivalent antibody/antigen complexes. As an amplification is understood every enzymatically mediated reaction serving for the replication of a nucleic acid, for instance the PCR.

### BACKGROUND OF THE INVENTION AND PRIOR ART

Nucleic acids serve in natural organisms mainly for coding proteins to be expressed in a cell and the like. This is determined by the primary structure of the nucleic acid, i.e. the sequence. Independent herefrom, antibody/antigen complexes may however also enter into bindings with non-nucleic acids existing in a cell. Whether such a binding may occur, depends not only upon the primary structure, but also upon secondary and tertiary structure generated in a solution for a defined sequence (three-dimensional structure). The affinity of the nucleic acid to the target molecule at last depends upon whether the nucleic acid—in addition to the purely chemical binding capability—“matches” in steric regard in the region of the binding site or the binding sites of the target molecule, corresponding to the conditions for classic antibody/antigen complexes. Matching nucleic acids may thus exert the function of an antibody or antigen. Such aptamers normally are non-natural nucleic acids and can “tailored” for a target molecule. For tailoring, there are in principle two approaches. The first approach is the calculation of a suitable sequence and/or derivatization for the nucleic acid according to the precisely known structure, including binding sites and tertiary structure, of the target molecule. This is not only extremely costly; in cases where the structure of the target molecule is not sufficiently known, this approach is impossible. The second approach consists in the isolation of the target molecule and in the contacting of a mixture of prospective nucleic acids with the isolated (and in most cases immobilized) target molecule, wherein nucleic acids that bind with high affinity are separated from those that bind with less affinity or do not bind at all. The mixtures of the nucleic acids are typically nucleic acid libraries, for instance, established by the combinatorial chemistry. A nucleic acid library contains a plurality of different nucleic acids, at least in a partial sequence region a randomization (with natural and/or non-natural nucleotides) is provided. A preserved sequence region may be provided, however it is not necessary. Randomization in n positions with m different nucleotides leads to a library with n^{m} elements. The selected high-affinity nucleic acids are suitable for a plurality of applications.

For instance, nucleic acids may be used in tests for the existence or non-existence of target molecules specific for the nucleic acid in a test solution and/or in a cell or tissue. Then the presence of a reporter molecule of a conventional structure in the nucleic acid for the easy identifiability by a measurement is recommended. Such tests may be used in diagnostics, for instance, the diagnostics for oncogenic mutants or marker substances resulting from certain physiological malfunctions. It is understood that the respective nucleic acid needs not only selectively “detect” the oncogenic mutant, but must not bind to the natural variant, i.e. must discriminate between an oncogenic expression product and a natural expression product. This can easily be performed after the selection of nucleic acids binding with the oncogenic expression product, namely by the subsequent selection of nucleic acids which do not bind with the natural expression product from the previously selected nucleic acids.

Selected nucleic acids may also be used for the separation of target molecules from a solution, for instance in conventional column or gel separation methods. Then it is recommended to have the nucleic acid immobilizable and to immobilize it in the separation method.

Selected nucleic acids may however also be used for modulating physiological functions, i.e. inhibiting, inducing or reinforcing. Such nucleic acids are thus also used in pharmaceutical compositions. In addition, selected nucleic acids have, of course, to be physiologically acceptable in order to avoid side effects. The advantage of using nucleic acids in lieu of, for instance, peptides, is that the identification or selection of suitable nucleic acids is considerably easier than in the case of the peptides or proteins because of the comparatively easy producibility with regard to protein or peptide libraries.

A known method for selecting nucleic acids that bind with high affinity to a target molecule is known as the SELEX method (Systematic Evolution of Ligands by EXponential enrichment). Various variants are for instance described in the documents U.S. Pat. No. 5,712,375, U.S. Pat. No. 5,864,026, U.S. Pat. No. 5,789,157, U.S. Pat. No. 5,475,096, U.S. Pat. No. 5,861,254, U.S. Pat. No. 5,595,877, U.S. Pat. No. 5,817,785. In the insofar known methods, in principle, approximatively in a plurality of cycles such nucleic acids are separated which bind with high or higher and higher affinity. In every cycle, the selected group must be amplified with nucleic acids. The separation of binding nucleic acids from the target in every cycle takes place by specific driving-out. This method has several drawbacks. First of all, it is disadvantageous that, due to the required number of cycles, a rather large amount of nucleic acids as well as of target molecules is necessary. Further, in the driving-out step, more (bound) nucleic acids of lower affinity are driven out from the bond than nucleic acids of higher affinity, thus the difference in amounts is increased at the expense of the higher-affinity nucleic acids in the amplification step. The difference in amounts is further increased by the fact that with higher affinity, the bond of the separated nucleic acids to the ligands used for the separation is comparatively stronger, and the higher-affinity nucleic acids are thus less accessible for the amplification. It is further disadvantageous that with increasing affinity of the nucleic acids, a logarithmically increasing concentration of the ligand used for the separation is required. The obtainable affinity is thus limited by the solubility product of the used ligands. Finally, it is disadvantageous to have to operate in several cycles for the repeated separation or selection of nucleic acids selected in a pre-cycle.

In the field of the separation of non-nucleic acids, the affinity chromatography, in particular as column chromatography (solid/liquid phase), is well known. It is a method for the isolation or enrichment, up to 10⁵ times and more, of for instance proteins. A ligand of the sequence to be enriched is immobilized at a chromatographic matrix. Highly affinitive compounds are firstly bound, i.e. at the entrance of the column. Downstream less affinitive compounds are bound, as far as the amount of the affinitive compounds, referred to the respective specific compound, is lower than the amount of ligands in the column. Weakly affinitive or not affinitive compounds will pass through and are thus separated from the affinitive compounds. Bound, i.e. affinitive compounds, are then eluted and further used. With non-specific desorption methods, for instance physicochemical or thermal methods, a mixture of differently highly affinitive (bound) compounds takes place. In the desorption with ligands of the bound compounds (driving-out desorption), a very high molar amount of the ligand is necessary for the separation in particular of the highly affinitive compounds.

Further, in general, the separation capacity is then not acceptable, if the target molecules are small or very small, for instance 50 to 10,000 Da, in particular 70 to 2,000 Da, and a derivatization for the improvement of the separation capacity is to be avoided. The structural heterogeneity of the nucleic acids of a combinatorial nucleic acid library makes difficult or impossible in this case the separation of the nucleic acid/target molecule complexes from the not-complexed nucleic acids of the nucleic acid library by a simple linear or one-dimensional separation.

### Technical Object of the Invention

The invention is based on the technical object to provide a method of selection of nucleic acids that bind with high affinity to a target molecule, which supplies with less expenses highly affinitive nucleic acids in particular against very small target molecules, without a derivatization of the target molecules being necessary.

### SUMMARY OF THE INVENTION

For achieving said technical object, the invention teaches a method of selection, by two-dimensional separation, of nucleic acids that bind to a target with high affinity from a mixture of nucleic acids, including the following steps: a) subjecting the mixture of nucleic acids to a physico-chemical separation step, thereby obtaining a set of mixed fractions containing the nucleic acids, a run parameter window being associated with every mixed fraction containing the nucleic acids, b) contacting a mixed fraction containing the nucleic acids with the target molecule, thereby obtaining a binding mixture containing nucleic acid/target molecule complexes, c) subjecting the binding mixture from step b) to the same physico-chemical separation step as in step a), thereby selecting nucleic acid/target molecule complexes whose run parameters are outside of the run parameter window.

As physico-chemical methods can in particular be used: electrophoresis (e.g. capillary electrophoresis, flat bed electrophoresis, horizontal electrophoresis) and the chromatography methods (e.g. solid/liquid chromatography, liquid/liquid chromatography, capillary chromatography). The detailed methods and reagents to be used herefor can easily be determined by the one skilled in the art according to the target molecules and/or nucleic acids. In physico-chemical separation methods, a separation of the applied substance mixtures takes place in fractions, a run parameter window being associated with each fraction. As run parameters, for instance time and/or travel can be used. A run parameter window contains an initial value and an end value of the run parameter, within which a fraction is gained. A fraction may contain one or several nucleic acid species. A binding mixture is a mixture of different nucleic acid/target molecule complexes. The term of the two-dimensional separation relates to the subsequent application of the same separation method on one hand of the nucleic acids and on the other hand of the complexes. This will become evident in the examples.

A single species of target molecules may be used, however several different defined or undefined species may also be used. The solution of the nucleic acids and of the target molecules takes place in the usual buffers, for instance Tris buffer or acetate buffer. Nucleic acid libraries are mixtures of nucleic acids with a number of typically 10⁶ to 10²²/mole, in particular 10⁶ to 10²¹/mole nucleic acid species separated from each other. In a used library, each nucleic acid species is statistically present for instance with 10 to 10¹⁷, in particular 10 to 10¹³ molecules. The binding of the nucleic acids to the target molecules preferably takes place under conditions corresponding to a later use of the nucleic acids, i.e. for instance in a buffer correspondingly adjusted with regard to temperature, ionic strength, pH value and buffer conditions. The solvent of the nucleic acid library is then to be correspondingly selected with regard to its components. The same applies to the solvent where the target molecules are dissolved.

The invention is based on the surprising finding that the binding of a target molecule to a nucleic acid has an influence on the behavior of the nucleic acid in a physico-chemical separation method. The influence is the higher, the larger the affinity constant of the binding is, i.e. the stronger the binding between nucleic acids and target molecule is. The invention is particularly suitable for the selection of nucleic acids against unmodified small target molecules, for instance 50 to 10,000 Da, in particular 70 to 2,000 Da. A chemical modification of very small target molecules makes difficult or impossible a selection of nucleic acids affinitive against the unmodified target molecule.

Subsequent selection artifacts at the expense of higher-affinitive nucleic acids are avoided. Ligands, in particular high concentrations of ligands, are not required for the desorption. Finally, virtually all bound and then desorbed nucleic acid molecules are available for an amplification. This permits to use low nucleic acid concentrations. In principle it is already sufficient if each species is present in the nucleic acid library by one molecule in a statistical average.

Nucleic acids isolated or nucleic acid mixtures produced with the method according to the invention (to be brief, nucleic acids) can be used in various ways. For a respective specific application it is only necessary to use target molecules selected according to the application in step b). It is for instance possible to identify marker substances being characteristic for a disease, to determine with the method according to the invention nucleic acids that bind with high affinity thereto, and to use the thus selected nucleic acids as a main component of a test kit for the investigation for the marker substance or for the presence or the risk of the disease. Such test kits may of course also be used for the therapy control. The nucleic acids may also be used for preparing pharmaceutical compositions, for instance when an inhibition of the marker substance (by the binding of the nucleic acid) leads to a reduction or prevention of the symptoms. By complexing little target molecules by highly affinitive nucleic acids, in addition an improved venal segregation in particular of unpolar targets can be achieved. It is also possible, by selection of the target molecules, to select nucleic acids and use them as a pharmaceutical composition, which stimulate the generation of substances missing in the case of a disease in an organism. By selection of suitable target molecules, for instance nucleic acids may also be found which influence as effective substances the differentiation and/or stimulation or suppression of isolated cells (for instance blood cells, such as T cells, granulocytes, monocytes or thrombocytes). The same may be effected for cells in an aggregate (tissue) and the like in the field of tissue engineering. Finally, nucleic acids selected according to the invention may for instance be used in an affinity matrix as an immobilized phase (for instance apheresis) or for the specific desorption of substances of an affinity matrix.

An essential element of the invention is the selection of nucleic acids binding to very small target molecules. These may substantially enclose the target molecules and thus prevent the elimination of the target molecules e.g. from the blood circulation.

### PREFERRED EMBODIMENTS OF THE INVENTION

Subsequently to step c) in most cases a separation step d) is performed, wherein the nucleic acids of selected nucleic acid/target molecule complexes are separated from the target molecules. After step b), if applicable after dissociation in step c) of the obtained nucleic acid/target molecule complexes, an amplification may be performed by means of for instance PCR, RT-PCT or LCR.

A dissociation (in a separation step) of nucleic acid/target molecule complexes obtained by the method according to the invention may for instance take place by driving-out with a sufficiently strong ligand, modification, complexing and/or destruction of the target molecule, physico-chemically or thermally. Mechanical methods, for instance ultrasonic methods may be used for the dissociation or strengthening of the dissociation. It is understood that the nucleic acids may not be decomposed by the employed method of dissociation. Preferably, the non-specific dissociation by means of usual physico-chemical or thermal methods is performed. Thermal dissociation is effected by heating the obtained solution. Heating may for instance be made by electrical heating or irradiation of microwaves or IR. In particular, the heating technologies of the PCR technology are suitable.

The non-specific desorption may be supported by a chemical modification of the target, e.g. oxidation by sodium periodate or the like, or by non-specific complexing, for instance by means of borate or the like for blocking cis-trans diol bonds in hydrocarbons. It is particularly preferred that the non-specific desorption is performed by a thermal desorption in a preferably extended high-temperature phase of a PCR or RT-PCR. Hereby a synergy effect is achieved, since normally, in particular when working with nucleic acid libraries of low concentrations of the nucleic acid species, an amplification is anyway required. The obtained nucleic acids (after the dissociation) can be amplified easily and without disturbing ligand couplings.

For increasing the yield, for instance 5 to 60, preferably 20 to 60, most preferably 45 to 55 cycles are used for the amplification, PCR, RT-PCR, LCR or the like. For the amplification, at least one marked primer can be employed. The primer may comprise at least one endonuclease interface. Such an interface serves for instance for freeing the amplificate from larger regions of the primer sequence. Nucleotide components in the primer or in the nucleic acids to be selected may be marked for instance by fluorescence dyes. As fluorescence dyes are mentioned for instance: Alexa® Fluor 488, Fluor 532, Fluor 546, Fluor 568, Fluor 594, Oregon Green 488, Fluorescein, Rhodamine 6G, tetramethylrhodamine, Rhodamine B and Texas Red. The amplificate may also be marked at different ends by two different chemical modifications, if the groups introduced by the modification can be bound as ligands respectively at a different affinity matrix.

It is preferred that the nucleic acids are functionalized for the physico-chemical separation methods. If as a physico-chemical method for instance an electrophoretic method is employed, the nucleic acids (of the various species) may be provided with a group carrying an electric charge.

In the following, the invention is explained in more detail, based on not limiting examples of execution.

### EXAMPLE 1

### Selecting by Run Travel Separation Methods

A nucleic acid library prepared in a conventional manner is applied on a suitable gel, e.g. agarose gel. After heating for melting any double-stranded nucleic acids, an electrophoretic separation is performed at 4° C., in one space dimension, the run travel. Then the gel is cut into stripes in the direction in parallel to the run travel, and one, several or all stripes are incubated with target molecules. A thus obtained stripe with complexes is brought on an identical second agarose gel, and the complexes of the stripe are transferred on the second gel by means of mechanical and/or physico-chemical methods. Then, under the same conditions, another electrophoretic separation is made, the run direction being orthogonal to the longitudinal extension of the applied stripe, i.e. in a second space dimension. Nucleic acids not having formed complexes then lie on the second gel on a diagonal. The second gel is cut into stripes, the longitudinal extension of which is orthogonal to that of the applied stripe. From a thus obtained stripe of the second gel, that region is separated which corresponds to the run travel window of the part associated with the stripe of the applied stripe. The remainder of the stripe is collected and subjected to a dissociation step and an amplification step.

### EXAMPLE 2

### Selecting by Run Travel Separation Methods

A nucleic acid library prepared in a conventional manner is applied on a suitable HPLC. Then a separation of the nucleic acids is performed, in one time dimension, the run time, with fractions being caught in defined run time windows. Such a fraction is then incubated in a suitable way with the target molecules. The incubated fractions are then separated again, under the same conditions, i.e. in a second time dimension. From the thus obtained fractions of the second HPLC, those fractions are rejected the run time of which corresponds to the run time of the applied fractions. The remaining fractions of the second HPLC are collected and subjected to a dissociation step and an amplification step. All fractions of the first HPLC are treated correspondingly.

1. A method of selection, by two-dimensional separation, of nucleic acids that bind to a target with high affinity from a mixture of nucleic acids, comprising the following steps:
a) subjecting the mixture of nucleic acids to a physico-chemical separation step, thereby obtaining a set of fractions containing mixtures of nucleic acids, a run parameter window being associated with every fraction containing the mixtures of nucleic acids,
b) contacting a fraction containing the mixture of nucleic acids with the target molecule, thereby obtaining a binding mixture containing nucleic acid/target molecule complexes, and
c) subjecting the binding mixture from step b) to the same physico-chemical separation step as in step a), thereby selecting nucleic acid/target molecule complexes whose run parameters are outside of the run parameter window.

2. A method according to claim 1, wherein subsequent to step c) a separation step d) is performed, wherein the nucleic acids of selected nucleic acid/target molecule complexes are separated from the target molecules.

3. A method according to claim 1 or 2, wherein the selected nucleic acids are amplified.

4. A method according to claim 1 or 2, wherein the nucleic acids are functionalized for the physico-chemical separation method.

5. Nucleic acids or nucleic acid mixtures obtainable by a method according to claim 1 or 2.

6. Test kit for the detection of marker substances characteristic for a disease, a functional condition or the risk of a disease or for the preparation of pharmaceutical compositions comprising the nucleic acid or nucleic acid mixtures according to claim 5.

7. A method according to claim 3, wherein the nucleic acids are functionalized for the physico-chemical separation method.

8. Nucleic acids or nucleic acid mixtures obtainable by a method according to claim 3.

9. Nucleic acids or nucleic acid mixtures obtainable by a method according to claim 4.

10. The kit of claim 6, wherein the selected nucleic acids are amplified.

11. The kit of claim 6, wherein the nucleic acids are functionalized for the physico-chemical separation method.
